(19) United States
(12) Patent Application Publication
(10) Pub. No.: US 2007/0294101 A1
(43) Pub. Date: Dec. 20, 2007

(54) METHOD AND SYSTEM FOR ENFORCING BUSINESS POLICIES

(76) Inventors: Sanjay Dalal, Milpitas, CA (US); Ramana Yerneni, Cupertino, CA (US); Sharad Thankappan, Belmont, CA (US)

Correspondence Address: HAHN AND MOODLEY, LLP, P.O. BOX 52050, MINNEAPOLIS, MN, 55402, US

(21) Appl. No.: 11/739,601

(22) Filed: Apr. 24, 2007

Related U.S. Application Data
(60) Provisional application No. 60/745,445, filed on Apr. 24, 2006.

(51) Int. Cl. G06Q 50/00

(52) U.S. Cl. 705/1

(57) ABSTRACT

In one embodiment, there is provided a method, comprising: reading an Electronic Product Code (EPC) from a carrier associated with a product; accessing a policy associated with the product; and performing a policy enforcement operation based on the policy, wherein the policy is securely downloaded from a server on the Internet that is authoritative for an Internet domain of a legitimate provider of the product.

This application claims the benefit of priority to U.S. Provisional Patent Application Number 60/745,445 filed on Apr. 24, 2006, which is incorporated herein by reference.

### FIELD

Embodiments of the invention relate to a method and a system for enforcing business policies.

### BACKGROUND

Most products travel through a complex supply chain from the point of production to the final consumer. This supply chain may include various entities such as raw material suppliers, manufacturers, wholesalers, distributors, logistics providers, and retailers. These entities would like to enforce business policies (hereinafter “policies”) that govern the manufacture, storage, handling, and sale of products as they travel through the supply chain. These policies may be enforced both within an entity's organization and by its supply chain partners. For example, policies can be defined to ensure that products are not diverted into illegal channels of distribution, that counterfeit products are not sold to consumers, and that the quality of the product is not compromised through poor storage practices.

Consider an example of product diversion in the pharmaceutical supply chain. The majority of drugs are sold by manufacturers to primary distributors. These distributors then sell these drugs to the point of dispensing (e.g. pharmacies, retail stores, and hospitals). This flow is referred to as the normal chain of distribution (NCOD). Often, however, drugs are sold at highly discounted prices directly to subsidized groups such as nursing homes or exported to other countries. Such drugs are usually restricted by law or contracts to be used solely within the markets they have been sold to. These lower-priced drugs are often illegally smuggled back (diverted) by secondary and tertiary distributors into the NCOD and sold to primary distributors for a profit. A drug bounces back and forth from distributor to distributor, creating a supply chain that is complex, convoluted, and vulnerable. the more frequently a drug changes hands, the greater the chance that counterfeit drugs can enter the NCOD—even more so when the distribution networks span over many countries. These illegal practices can result in significant loss of revenue, compromised customer safety, and damage to brand integrity.

Some states have issued regulations that require each party engaged in the sale of drugs to provide a pedigree document to the purchaser. A pedigree is a certified record that contains a paper trail about the distribution of a drug. It records the sale of an item by a pharmaceutical manufacturer, any acquisitions and sales by distributors or repackagers, and final sale to a pharmacy or other entity dispensing the drug. Requirements for pedigree would certainly reduce drug diversion and reduce possibilities for counterfeit. However, creating and maintaining paper-based pedigrees for a large volume of drugs is expensive, error-prone, time-consuming, and susceptible to fraud. So, paper-based, manually created pedigrees have not worked in practice.

Pharmaceutical companies are working to create electronic pedigrees (ePedigree) for drugs using technologies such as RFID and EPC. RFID is an electronic identification technology that uses radio-frequency signals to read information from tags that are attached to physical objects. These tags often have an associated EPC. EPC is a global standard that is used to assign unique serial numbers to objects. It incorporates a hierarchical structure that can be used to express a wide variety of different, existing numbering systems like the EAN.UCC System Keys, UID, VIN, ISBN, and so on. Passive RFID tags can be attached to drug vials. An EPC inserted into each RFID tag can associate a unique serial number with each drug ial and thereby provide unique identification.

The RFID tag can be read automatically as unopened boxes pass by electronic readers installed at various entities within the supply chain. This enables electronically creating tracing logs for each vial sighted by RFID readers. These logs effectively provide an electronic pedigree. however, as the number of products moved across the NCOD is so large that the volume of these logs could easily overwhelm the current track and trace software infrastructure. Moreover, additions to the pedigree made by downstream supply chain participants are not automatically visible to all the upstream participants. This is particularly disadvantegeous to manufacturers, who suffer the most business loss due to diversion, as they do not get visibility into the flow of drugs beyond the first point of distribution.

Some solutions propose a centralized, hosted service approach to collect and disseminate tracing data from all supply chain partners. this should theoretically enable upstream participants to gain complete visibility. There are various business and technical hurdles to this approach. Supply chain partners may be unwilling to centrally share data for security and privacy reasons. Availability, scalability, and other technical infrastructure requirements can impose significant costs as well.

Also, to effectively counter diversion, all parties in the supply chain must agree to install RFID systems. For example, if a secondary or tertiary distributor does not install RFID infrastructure, the ePedigree will be incomplete. Getting buy-in from all participants can be a long-drawn process. Such lack of buy-in can further slow down RFID adoption since companies cannot get immediate benefits in terms of preventing (or reducing) product diversion.

### SUMMARY

In one embodiment a method, comprising:

reading an Electronic Product Code (EPC) from a carrier associated with a product;

accessing a policy associated with the product; and

performing a policy enforcement operation based on the policy, wherein the policy is securely downloaded from a server on the Internet that is authoritative for an Internet domain of a legitimate provider of the product.

### BRIEF DESCRIPTION OF THE DRAWINGS

FIG. 1 the architecture of a system for enforcing business policies, in accordance with one embodiment of the invention;

FIG. 2 shows the operations for policy authoring, in accordance with one embodiment of the invention;

FIG. 3 shows the operations for policy provisioning in accordance with one embodiment of the invention; and

FIG. 4 shows the operations for policy enforcement, in accordance with one embodiment of the invention;

### DETAILED DESCRIPTION

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

Reference in this specification to “one embodiment” or “an embodiment” means that a particular feature, structure, or characteristic described in connection with the embodiment is included in at least one embodiment of the invention. The appearance of the phrase “in one embodiment” in various places in the specification are not necessarilty all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

For descriptive purposes, assume that company “C” that produces drug “D” has shipped a batch of vials to country “A”. These vials have RFID tags with EPC serial numbers ranging between “n1” to “nn”. However, they observe that drugs shipped to country “A” at discounted prices are smuggled back into the more lucrative market of country “U” through illegal channels consisting of secondary and tertiary wholesalers. “C” could author a simple policy to detect such diverted items in country “U”. An English language description for this policy could be “If serial number of EPC falls between range “n1-nn” and item is drug “D” produced by company “C” and current location is “U” then report diversion”. “C” could make this policy available to other business entities in the distribution chain of “D” in the country “U” for enforcement. Policy enforcement may result in the execution of one or more actions such as notifying appropriate parties of violation as well as auditing the decision.

Since policies specify general business rules, they avoid the issues (outlined earlier) of sharing large quantities of data associated with tracing the flow of each item in the supply chain. In addition, they eliminate the need for every participant in the supply chain to participate in the solution to guarantee efficacy. For example a pharmacy could enforce the above policy directly and detect diversion, even if other intermediaries like secondary distributors have not done so.

In one embodiment, there is provided a method and system (also referred to herein as “technology”) for enforcing a business policy that allows entities to author machine-readable policies and then securely and reliably distribute these policies over the Internet to other authorized business entities. The technology enables businesses to enforce those policies electronically at the right place and time in the supply chain using RFID and EPC technologies. Depending on the problem being addressed, the authorized sources for policies could be manufacturers of the products, regulatory agencies, or some other entity. In addition to enforcing policies electronically, it is also possible to feed back to the authorized sources the exceptions or violations observed during policy enforcement. This could help policy authors to enhance existing policies or create new ones.

In one embodiment, the technology may be implemented in supply chains that are multi-tiered and non-deterministic. The technology enables policy authors to distribute policies to entities with whom no direct, pre-existing relationship exists. Further, the technology achieves this without creating any data sharing related business (e.g. data confidentiality) and infrastructure problems.

Another advantage of the technology is that it provides visibility to the upstream participants (e.g. manufacturers) and regulatory agencies in case of violations. The technology distributes only rules or policies with minimal data to participants in the supply chain. It does not require collecting and/or processing huge amounts of product trace data centrally to detect diversion. It would be a more acceptable solution to users concerned with business and technical issues related to data sharing and ownership.

Finally, it does not require implementation by every participant in the supply chain to be effective.

In this description, Radio Frequency Identification (RFID) and Electronic Product Code (EPC) based solutions that will enable manufacturers to achieve the above goals, are described.

However, one skilled in the art would appreciate that the technology provides an extensible framework to author, to provision, and to enforce policies for EPC-tagged product items as well as to report and audit violations of these policies, regardless of the particular carrier of the EPC. The technology may be used to enforce policies for various purposes including, but not limited to illegal product diversion detection, counterfeit detection, recall determination, and material handling.

1.1 Architecture

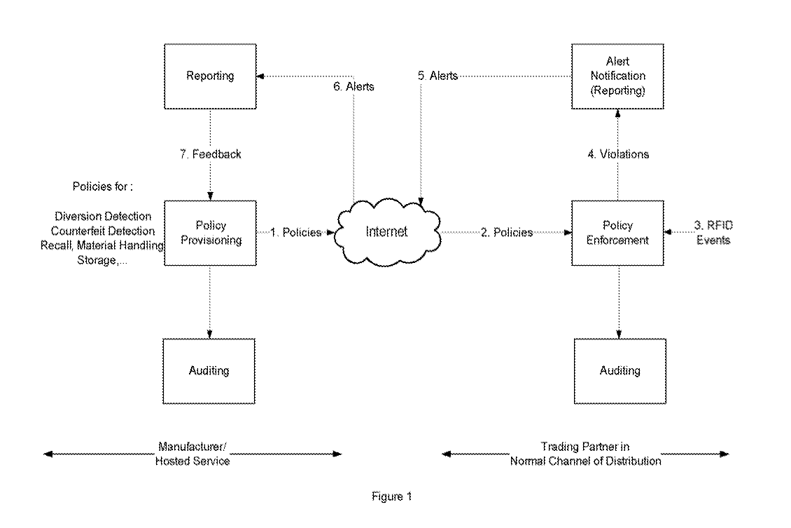

The main functional components of technology are policy Provisioning, Policy Enforcement, Reporting and Auditing. FIG. 1 shows a simple configuration involving these elements.

An EPC Policy is applied to a resource. For example, an RFID tagged vial of drug “D” identified by an EPC is a resource. Typically, an EPC Policy consists of one or more rules where rules define the criteria for resource access and usage. A rule is a binding of a set of actions to a set of conditions. The conditions are evaluated to determine whether the actions must be performed.

The Policy Provisioning component is where authoring and provisioning of EPC Policies takes place. This component would typically have a Master Policy Store that maintains one or more versions of policies and related data. The Policy Provisioning component is owned by an authorized policy source in the supply chain—e.g. manufacturer “C” would provision policies for drug “D”. Policies are then distributed securely as and when needed to Policy Enforcement components.

The Policy Enforcement component is the point at which policy decisions are actually enforced. policy Enforcement components could be spread across the Internet and owned by different parties—e.g. distributors and pharmacies could run Policy Enforcement components for drug “D”. Each component runs on a policy-aware node. A policy-aware node could be co-located with Policy Provisioning component or it could be remotely located. it could be running on an RFID edge server, a network node such as a router or a switch, an appliance or an enterprise server, among other things. To enforce a policy, the Policy enforcement component could either contact the Policy Provisioning component or a local proxy that could cache policies and related data for performance reasons. The cache could also be backed up by a Local Policy Store that can be synchronized with the Master Policy Store using various configurations such as periodic sync, time to live, etc.

Certain steps performed while Provisioning and Enforcing of policies are potential candidates for non-repudiation when multiple parties are involved. Non-repudiation provides protection against false denial of involvement by any party that has provisioned or enforced a policy. The technology provides an audit service that records information needed to establish accountability for actions taken during the provisioning and enforcing processes.

The technology provides a Reporting capability that alerts appropriate parties when violations occur during policy enforcement. In addition to notifying systems and users enforcing policies, the policy authors can also be notified. for example, the drug manufacturer “C” would be interested in knowing if a batch of items with EPC serial numbers in the range “n1-nn” that it had sent for charity purposes to country “A” entered the distribution channel in country “U” due to illegal diversion.

1.2 Process

In one embodiment, the technology involves three main processes: Provisioning, Enforcement, and Reporting

1.2.1 Provisioning

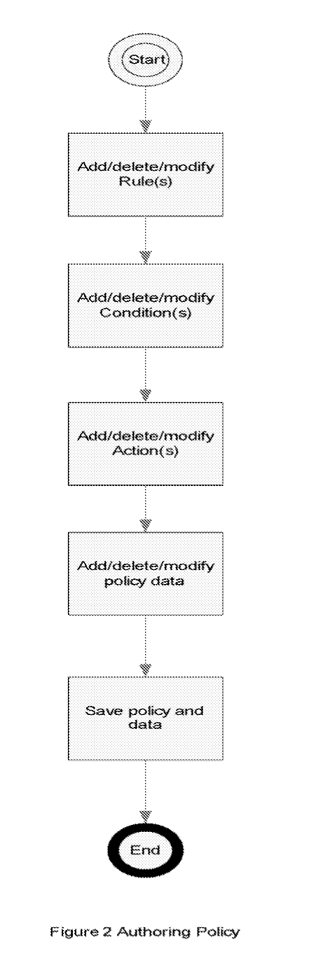

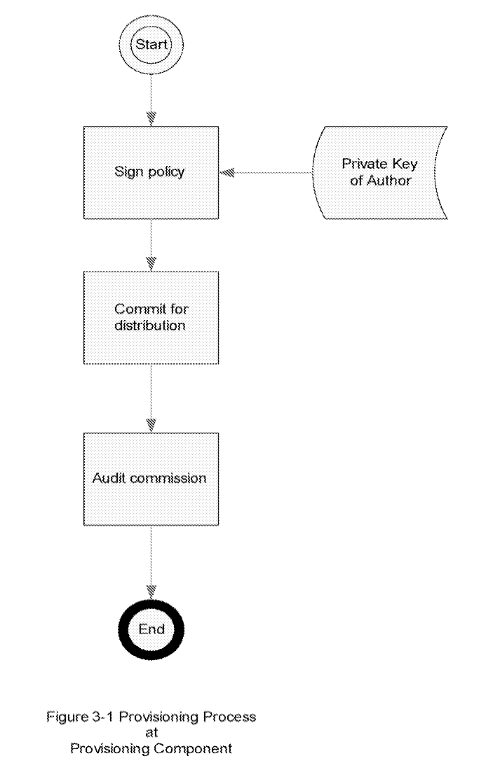

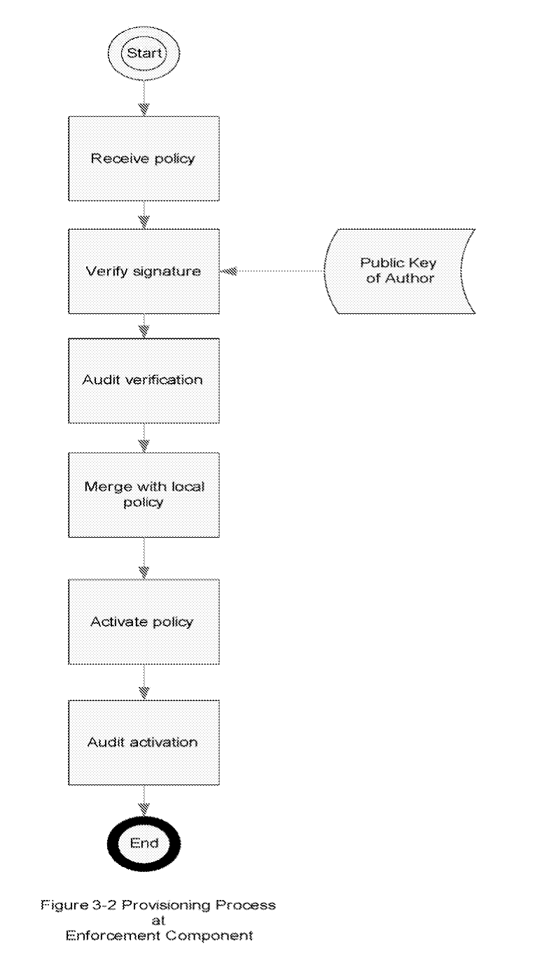

Policy authoring is centralized and policy enforcement is distributed in the technology. The logical steps for policy authoring process are described in FIG. 2. For interoperability and readability reasons, the technology policies are described in XML using standard policy language (with extensions if required) such as one described in OASIS XACML. Once the EPC Policy is ready to activate, the Policy Provisioning process can push policies and associated data (as and when added/modified) securely to various Policy Enforcement components. Alternately, policies can be made available by the Policy Provisioning components to be pulled by the Policy Enforcement components using criteria such as time of the day, on systems restart, at pre-determined checkpoints, or optionally as per user-defined parameters. FIG. 3 shows logical steps involved in policy provisioning process at Provisioning Component (FIG. 3-1) and Enforcement Component (FIG. 3-2). here we assume that policy documents could be signed using XML Digital Signature or equivalent process and that the propagation of policies from Provisioning to Enforcement components is over a secure channel using Secure Sockets Layer (SSL). The commit of EPC Policy for activation at Provisioning component and activation of policy at the Enforcing Component are important actions that need to be reported to Audit services for non-repudiation purposes.

Pushing policies to Policy Enforcement components deployed on nodes in a dynamic and undetermined supply chain may be difficult if it is not possible to know of all destinations in advance. In this case, policies can be pulled, provided a URI of the Policy Provisioning component is made available.

If the URIs to retrieve EPC policies are made publicly available, one can manually configure the Policy Enforcement component to retrieve the policies and related data from these URIs. However, any such approach must ensure that the URIs are authoritatively bound to the source (usually this source is the “Company Prefix” available inside each EPC) For example, any illegal distributor must be prevented from masquerading as the manufacturer “C” of drug “D” and distributing policies purported to be from the manufacturer. Also, these URIs must be published in some standard way at pre-defined locations.

The technology provides an approach where the policy author can use the Internet's Domain name Service (DNS) to make this URI available over the Internet. Here, the author of the policy (e.g. manufacturer), could register URI for the Policy Provisioning Component against a Service name “EPC+Policy” into an NAPTR resource record of the Domain Name Service under his/her domain. This approach binds the URI with authority as only the domain owner (in this case the manufacturer) could perform this act.

The technology provides an algorithm to safely download policies. This algorithm uses the “Company Prefix” (or equivalent) found on the EPC of an RFID tagged item as a search key to search the onsepc.com domain space in DNS and retrieve the domain name of the authorized source of policy. Then it retrieves the URI from the NAPTR record with the service name “EPC+Policy”. It then uses this URI to securely download, cache and store either all policies and associated data or the ones specific to the “Item Reference” (or equivalent) found on the EPC. Note that this process need not be followed every time an RFID event with EPC is processed, rather only when policies from an authorized source for a product item need to be updated in the local store or on demand if policies are not found locally.

1.2.2 Enforcement

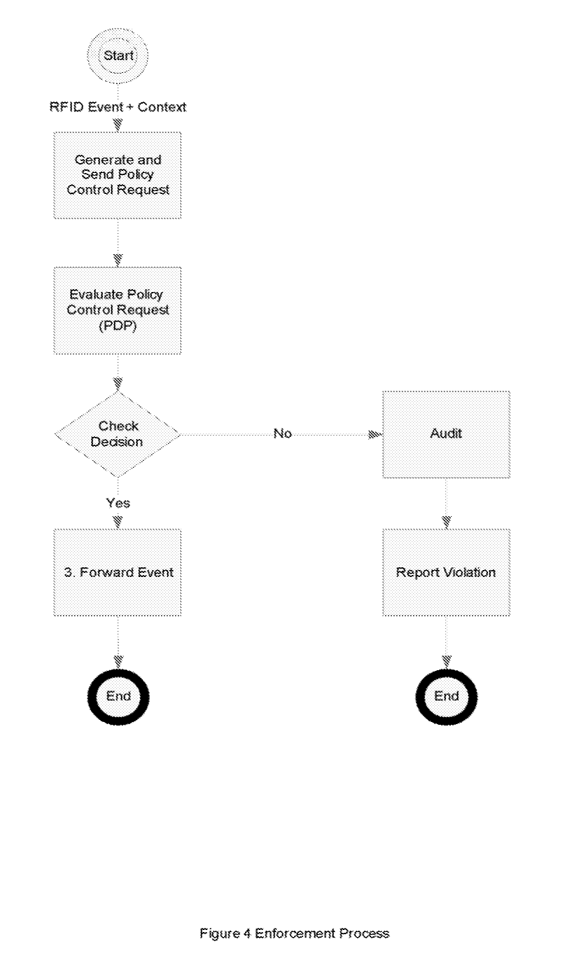

RFID readers generate RFID read events when they detect the presence of an object with an RFID tag. These events can be passed on to the Policy Enforcement component. Within the Policy Enforcement component, a Policy Control Request (PCR) is generated upon receipt of each such event. This PCR may contain the EPC representing the physical object along with additional contextual data. The event context could contain information about the environment in which the event is captured or processed, including but not limited to time of the event occurrence, locale where it occurred, identity owning the Policy Enforcement component, etc. The PCR is sent to a Policy Decision Point (PDP). The PDP retrieves the relevant policies to be enforced from the cache or policy store. For each retrieved policy, the PDP evaluates the policy condition. One example of EPC Policy condition could be “Does Company “C” approve distribution of its product “D” with EPC serial number range “n1-nn” in locale “U”!”. PDP then returns a policy decision that could be as simple as “true or false” or “allow or deny”. policy decision is then enforced by taking further actions such as notifying of a policy violation by raising an alert event and raising an audit event with the Audit service for non-repudiation purposes. logical steps involved in Policy Enforcement as described above are shown in FIG. 4.

1.2.3 Reporting

The technology provides a feedback path to the policy authoring entity (e.g. a manufacturer or regulatory agencies) by reporting violations of provisioned policies. This feedback is implemented through a Reporting Component. The policy author could make the URI of the Reporting component publicly available to enable policy-aware nodes to report violations. Policy-aware nodes hosting Enforcement components could then be pre-configured with the URI. Alternately, the URI could also be provided along with data and policies retrieved from the Provisioning components. The reporting format of how violations must be reported could be agreed upon by industry participants.

We have used an example of the pharmaceutical industry supply chain to describe the problem of product diversion and explain our solution. however, the technology is not specific to detecting product diversion in the pharmaceutical supply chain. It can be used detect product diversion in any industry where this problem may be present.

Moreover, the technology is quite extensible and flexible to implement various other kinds of policies including but not limited to product counterfeit prevention, product recall handling, product expiration checks, material handling, storage requirements, and environment monitoring.

The reach of Internet is across the world, therefore DNS is accessible everywhere. For simplicity, we have deliberately ignored delving into the security related export control regulations that needs to be followed.

Also, we have implicitly described the solution for a single type of electronic code (EPC) and a single type of carrier—the RFID tag, but, the technology does not assume this link between the EPC and its carrier. It can be used with other carriers including and not limited to bar code as well as other electronically readable product codes.

The URI resolution processes described to find URIs of Provisioning and Reporting components do not preclude using any already available URI from an NAPTR records. For example, there could be a pre-existing URI for EPC Information Service in an NAPTR record in the DNS domain owned by a policy author. It is possible to use such a URI to retrieve EPC Policies, and associated data including Reporting URI.

1. A method, comprising:
reading an Electronic Product Code (EPC) from a carrier associated with a product;
accessing a policy associated with the product; and
performing a policy enforcement operation based on the policy, wherein the policy is downloaded from a server on the Internet that is authoritative for an Internet domain of a legitimate provider of the product.

2. The method of claim 1, wherein the legitimate provider is selected from the group comprising an authorized supplier, distributor, and manufacturer of the product.
